(19) United States
(12) Patent Application Publication
(10) Pub. No.: US 2007/0260322 A1
(43) Pub. Date: Nov. 8, 2007

(54) Transversal support tibial plateau (TTTP)

(76) Inventor: Andrej Nowakowski, Salzkotten (DE)

Correspondence Address: Lipsitz & McAllister, LLC, 755 MAIN STREET, MONROE, CT, 06468, US

(21) Appl. No.: 11/788,779

(22) Filed: Apr. 19, 2007

Related U.S. Application Data
(63) Continuation of application No. PCT/DE05/01853, filed on Oct. 18, 2005.

(30) Foreign Application Priority Data
Nov. 3, 2004 (DE) 10 2004 053 075

(51) Int. Cl. A61F 2/38

(52) U.S. Cl. 623/20.32

(57) ABSTRACT

A transversal support tibial plateau consisting of two joint surfaces, two joint surface supports and a transversal support is described. This transversal support tibial plateau is arranged in such a way that the joint surfaces can be introduced separately from the front into the knee joint. This arrangement simplifies the surgical procedure, while the ventral corticalis is preserved. Owing to the construction with separate joint surface supports and joint surfaces, the joint surface supports can first be secured on the transversal support, for example, by means of support screws. Alternatively, the joint surface supports or the joint surface units can first be implanted obliquely from above with preservation of the ventral corticalis and then be received and supported from the side by a transversal support of corresponding design.

This application is a continuation of international application number PCT/DE2005/001853 filed on Oct. 18, 2005.

The present disclosure relates to the subject matter disclosed in international application number PCT/DE2005/001853 of Oct. 18, 2005 and German application number 10 2004 053 075.0 of Nov. 3, 2004, which are incorporated herein by reference in their entirety and for all purposes.

### BACKGROUND OF THE INVENTION

The invention relates to a transversal support tibial plateau (TTTP) consisting of two joint surfaces, two joint surface supports and a transversal support, so that the joint surfaces can be introduced separately from the front into the knee joint. In a further embodiment, the two joint surfaces with the two joint surface supports can form two joint surface units which, after insertion from above, can be received and supported by the transversal support from the side.

The advantages of the arrangement of a tibial plateau according to the transversal support principle are disclosed in WO 02/41809. It is known that implantation of a knee prosthesis with preservation of both cruciate ligaments places increased demands on the surgeon owing to the knee joint being openable to a lesser extent during the operation when the anterior cruciate ligament is left. Whereas a lateral approach with tuberosity osteotomy does not require the ventral corticalis to be worked on any further for introduction of the joint surface supports, the surgical procedure with the more often used medial approach is difficult and complicated.

The object of the invention is to create a transversal support tibial plateau which to the greatest possible extent meets the requirements of an implant according to the state of the art. Also, the implantation is to be simplified by corresponding arrangement of the components.

### SUMMARY OF THE INVENTION

This object is accomplished in accordance with the invention by a transversal support tibial plateau consisting of two joint surfaces, two joint surface supports and a transversal support, so that the joint surfaces can be introduced separately from the front into the knee joint.

The fundamental idea consists in first aligning and securing two single joint surface supports on an inserted transversal support on the tibia side. The joint surface supports can be introduced medially from above after outward rotation of the tibia relative to the femur or laterally after inward rotation.

Subsequently, the joint surfaces can be separately introduced directly from the front into the knee joint and secured and supported on the joint surface supports. This procedure makes it possible to maintain the complete ventral corticalis, even when a medial approach is used.

Alternatively, in a further embodiment, the joint surfaces can form with the two joint surface supports two joint surface units which, after insertion from above, are received and supported by the transversal support from the side. This can be implemented either by passing the transversal support through corresponding recesses in the joint surface units or by the support enclosing the bottom ends of the joint surface units, these being shaped accordingly. A correct permanent alignment and support can be achieved by a form-fitting design of the connection, for example, by means of guides or additionally by screws or bolts that are to be inserted ventrally.

The invention will be explained hereinbelow with reference to embodiments.

### BRIEF DESCRIPTION OF THE DRAWINGS

FIG. 1 shows schematically a transversal support tibial plateau according to an embodiment of the invention.

FIG. 2 (a, b) shows schematically a further embodiment of a transversal support tibial plateau according to the invention.

FIG. 3 shows schematically a front view of an embodiment according to the invention with a further embodiment of the joint surfaces and joint surface supports.

FIG. 4 shows schematically a lateral section of an embodiment according to the invention with a further embodiment of the joint surfaces and joint surface supports.

### DETAILED DESCRIPTION OF THE INVENTION

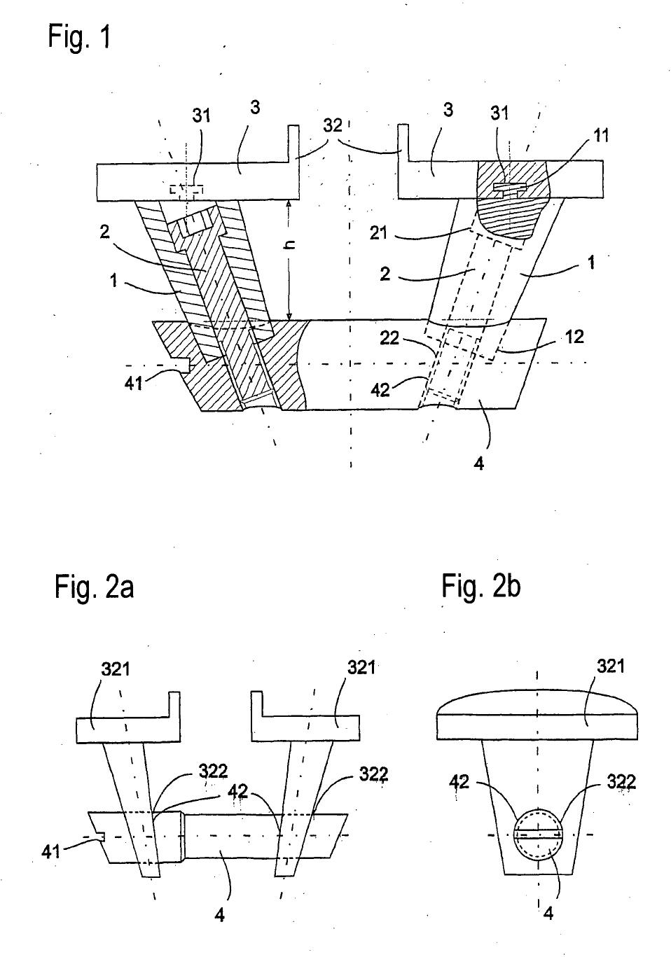

FIG. 1 shows a transversal support (4) on which two joint surface supports (1) are secured by means of support screws (2).

The joint surface supports (1) each receive, in turn, a joint surface (3). For this purpose, the recesses (31) are formed under the joint surfaces (3) in such a way that they can be introduced from the front via a corresponding guide (11) of the joint surface supports (1) and secured. A central position over the transversal support can be ensured by corresponding design of this connection with a stop or snap-in mechanism. According to this embodiment, the joint surface supports (1) are of conical construction, the bottom end (12) being received in a recess (42) of the transversal support (4). To achieve correct rotational alignment of the joint surface supports (1) which herein extend at an angle to the joint surfaces (3), an additional groove or guide can be provided between the bottom end (12) of the joint surface supports (1) and the recess (42) in addition to the screw connection between the recess (42) and the support screw thread (22).

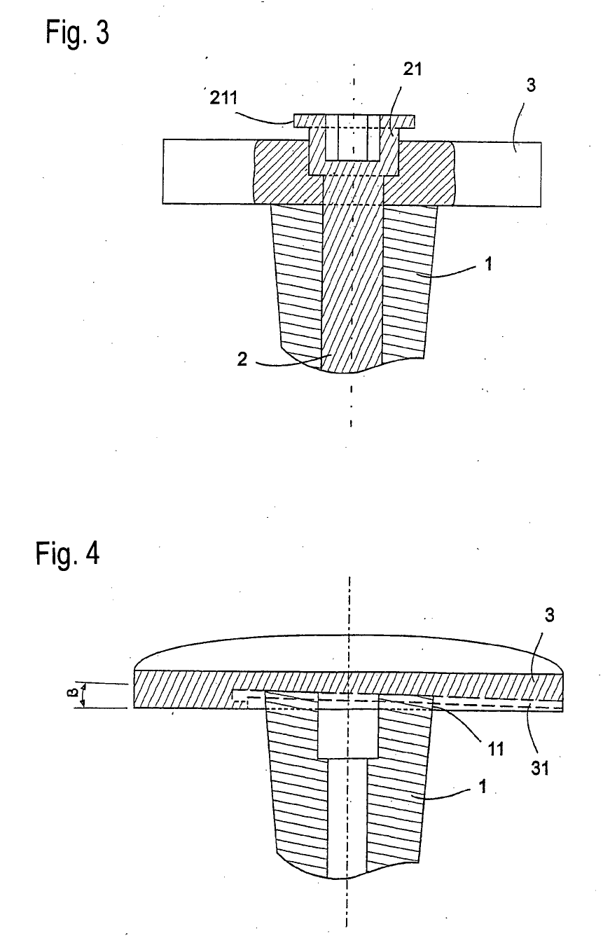

In this embodiment, the support screw head (21) remains within the joint surface supports (1). It can also be used additionally for guiding the knee prosthesis friction bearings (FIG. 3). Herein a stop (32) is formed to limit movement of the friction bearings. The height (h) between the joint surfaces (3) and the transversal support (4) is set to a constant by the screw connection (22, 42). A certain pressure can be exerted on the bone by means of further special configurations of the guides (11, 31), which increases the primary stability and promotes the osseous integration. Simpler connections in the form of form-fitting plug-in connections between joint surface support (1) and transversal support (4) are, however, also conceivable. In addition to a key surface (41) for insertion and alignment of the transversal support (4), the latter can have slanted end faces corresponding to the anatomical shape of the tibia.

FIG. 2a shows two joint surface units (321) which are supported by a transversal support (4). To facilitate insertion of the transversal support (4) via the key surface (41) after implantation of the joint surface units (321), the two recesses (322) with the associated bearing surfaces (42) can have different dimensions.

FIG. 2b shows a side view of FIG. 2a, in which the recesses (322) of the joint surface units (321) are represented as round bearings with bearing surfaces (42) of a cylindrical transversal support (4). Herein further form-fitting connections, such as threads, additional bolts, form-fitting embracing of a transversal support (4) of, for example, U-shaped construction, etc. are conceivable.

A further connection between joint surface (3) and joint surface support (1) is represented in FIG. 3. The joint surfaces (3) are secured by means of the support screw (2), whereby, with a suitable construction, the height (h, FIG. 1) can also be used in a positive manner to exert a corresponding pressure on the bone.

By means of a corresponding friction bearing guide (211), the support screw head (21) can also simultaneously serve to guide or attach the friction bearings of the knee prosthesis.

FIG. 4 shows a further connection between joint surface (3) and joint surface support (1). The height (h, FIG. 1) can also be influenced in a positive manner by the construction of the form-fitting connection (11, 31) rising at an angle (β).

1. A transversal support tibial plateau comprising:
a transversal support,
two joint surface supports, and
two joint surfaces,
wherein:
the joint surfaces are separately securable to the joint surface supports from a front side of the joint surfaces, and
the joint surface supports are connected in a form-fitting manner to the transversal support.

2. A transversal support tibial plateau in accordance with claim 1, wherein the joint surface supports are attached to the transversal support by support screws.

3. A transversal support tibial plateau in accordance with claim 1, wherein the joint surface supports are angled, and additional guides are provided for preventing rotation of the joint surface supports.

4. A transversal support tibial plateau in accordance with claim 2, wherein the joint surfaces are also secured to the joint surface supports by the support screws.

5. A transversal support tibial plateau in accordance with claim 2, wherein the support screws have heads which comprise slide-type guides for guiding or securing friction bearings of a knee prosthesis.

6. A transversal support tibial plateau in accordance with claim 1, wherein connections between the joint surfaces and joint surface supports are formed at an angle (β).

7. A transversal support tibial plateau in accordance with claim 1, wherein stops or snap-in mechanisms are provided for ensuring a position approximately at the center over the transversal support.

8. A transversal support tibial plateau in accordance with claim 2, wherein connections between the support screws and transversal support are assumed directly by the joint surface supports.

9. A transversal support tibial plateau in accordance with claim 2, wherein plug-in connections are formed between the joint surface support and the transversal support.

10. A transversal support tibial plateau in accordance with claim 8, wherein the connection of the joint surface supports to said transversal support in said form-fitting manner is rotationally stable even if the joint surface supports are not aligned perpendicularly to the joint surfaces.

11. A transversal support tibial plateau in accordance with claim 1, wherein the joint surface supports comprise recesses or guides at a bottom end thereof to enable the transversal support to be introduced from a side.

12. A transversal support tibial plateau in accordance with claim 1, wherein the joint surface supports comprise recesses or guides at a bottom end thereof to enable the transversal support to be pushed over from a side for securing purposes.

13. A transversal support tibial plateau in accordance with claim 12, wherein the recesses comprise round bearings.

14. A transversal support tibial plateau in accordance with claim 11, wherein the joint surface supports form a joint surface unit with the joint surfaces.

15. A transversal support tibial plateau in accordance with claim 11, wherein where the connection between the joint surface supports and the transversal support are secured by screws or bolts for additional locking.

16. A transversal support tibial plateau in accordance with claim 1, wherein separate connections between the joint surface supports and the transversal support have different dimensions to enable easier insertion of the transversal support using a corresponding auxiliary tool at a key surface or a flange.

17. A transversal support tibial plateau in accordance with claim 1, wherein the transversal support comprises a key surface for insertion and alignment of the transversal support with respect to the joint surface supports.

18. A transversal support tibial plateau in accordance with claim 1, wherein the transversal support has end faces which are slanted in accordance with an anatomical shape of the tibia.

19. A transversal support tibial plateau in accordance with claim 1, wherein the transversal support is of U-shaped or cylindrical design.
